(19) United States
(12) Patent Application Publication
(10) Pub. No.: US 2024/0206654 A1
(43) Pub. Date: Jun. 27, 2024

(54) Rechargeable waterproof smart cutlery and its charging case

(71) Applicant: SLOW CONTROL, L'sle adam (FR)

(72) Inventors: Jacques LEPINE, L'isle adam (FR); Guillaume DEJOUX, L'isle adam (FR)

(21) Appl. No.: 18/445,555

(22) PCT Filed: Feb. 28, 2022
(86) PCT No.: PCT/EP22/54917

§ 371 Date: Oct. 16, 2023

(30) Foreign Application Priority Data
Mar. 1, 2021 (FR) FR2101948

(51) Int. Cl. A47G 21/02

(52) U.S. Cl. CPC A47G 21/02; A47G 2200/08; A47G 2200/20

(57) ABSTRACT

Intelligent cutlery to guide the user in their feeding speed produced by the assembly of a cutlery head (1) of a cutlery handle (2) mechanically assembled with a protective case (30) of a card electronic (10) which comprises two power supply terminals, contact springs (21, 22) respectively make contact between the utensil head (1) and the Plus contact pad of the card and between the utensil handle (2) and the contact area Less of the card characterized in that the protective case (30) is sealed by a tamper-proof closure, the power supply is housed in the case (30) is a super rechargeable capacity, the utensil is recharged in a case by positioning by means of a timing circuit in contact the utensil head to the Plus terminal of an external power supply and the utensil handle to the Minus terminal of this external power supply.

The present invention relates to a rechargeable waterproof smart cutlery and its recharging case.

By intelligent, we mean a specific electronic device which 1—helps to eat slowly by warning the user if he eats too quickly 2—which allows recharging.

By cutlery we mean a fork, a spoon or a chopstick.

The present invention proposes a solution which allows 1—machine washing of the intelligent cutlery 2—rapid recharging of the power supply of its electronic circuit without compromising sealing 3—an optimized manufacturing cost price.

Cutlery needs to be washed at each meal. Enabling the intelligent nature of the utensil requires regularly recharging the battery which powers the electronics which control the intelligence of the utensil. There is no intelligent utensil solution in the prior art that is waterproof. There are smart cutlery solutions with battery or wired charging. There are in particular FR 0405342 and FR 0604201 which do not offer a solution for waterproofing. The problem with integrating an external power source to repower the utensil is that it requires frequent access to the terminals of the intelligent circuit to be able to recharge it. This access to the circuit terminals makes the sealing extremely complicated and therefore too expensive to be able to be integrated into a table utensil under acceptable cost conditions.

The three main constraints posed by a rechargeable intelligent table utensil are 1—Hold in a reduced mechanical volume 2—Be washed regularly 3—Be used regularly and therefore recharged regularly.

It is therefore understood that to solve this problem, it is necessary to design an optimized electronic circuit which fits particularly well with the mechanical elements. The invention therefore meets a double electronic and mechanical constraint to successfully achieve its objective.

It is also understood that the mouthing measurement is a measurement which is taken during the meal and which with the variety of users and the variety of meal conditions situations is subtle, rapid and versatile. This measurement must be able to be effectively detected and filtered by the electronic device.

In a main aspect, the invention proposes an intelligent circuit which comprises an electronic circuit comprising a charging circuit, a detection circuit, a warning circuit coupled to a super capacitor housed in a waterproof box with two terminals in contact on the one hand with the cutlery head and on the other hand with the cutlery handle.

In one aspect of the invention, the two parts of the cutlery each cooperate in two ways with the electronic circuit: For the cutlery head 1—to be the mouthing detection probe 2—to be the recharging terminal More of the circuit. For the utensil handle 1—to be the detection reference of the circuit 2—to be the charging terminal Less of the charging circuit.

It is therefore understood that the invention proposes a new six-fold cooperation between the electronic circuit and the two parts of the utensil, namely: 1—mechanical cooperation so that the assembled assembly has excellent mechanical strength 2—cooperation mechanical so that the two terminals of the circuit are in electrical contact with the two parts of the utensil 3—mechanical cooperation so that the whole constitutes a sealed assembly 4—electronic cooperation so that the whole detects mouthing events 5—electronic cooperation for the same assembly is recharged through the same terminals which allow detection 6—double mechanical-electronic cooperation of the assembly with a box for recharging the utensil.

The invention will be better understood on reading the appended figures in which:

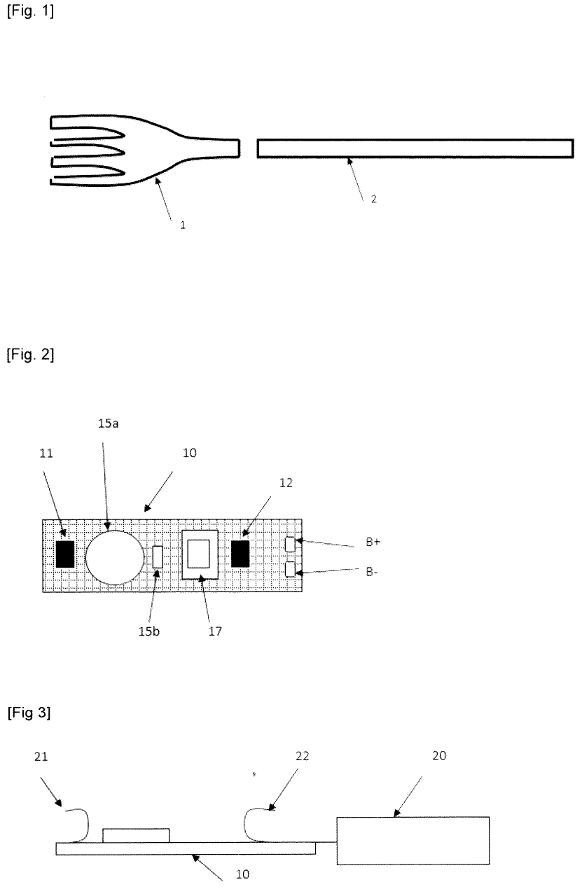

FIG. 1 represents a view of the two parts of the utensil

FIG. 2 represents a top view of the electronic card

FIG. 3 represents a side view of the electronic card

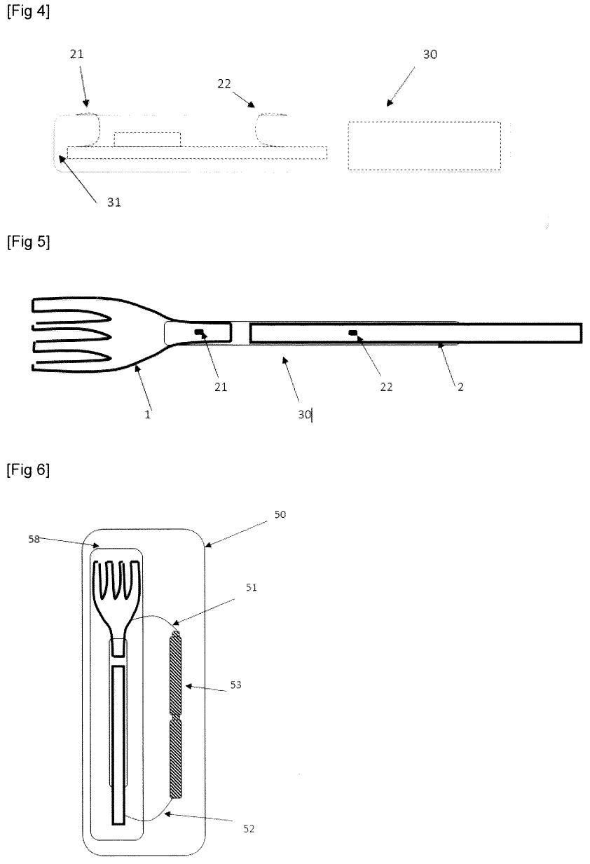

FIG. 4 represents a side view of the electronic card inscribed in its protective casing

FIG. 5 represents the assembly of the electronic card in its case with the two utensil parts

FIG. 6 represents the cutlery in its reloading case

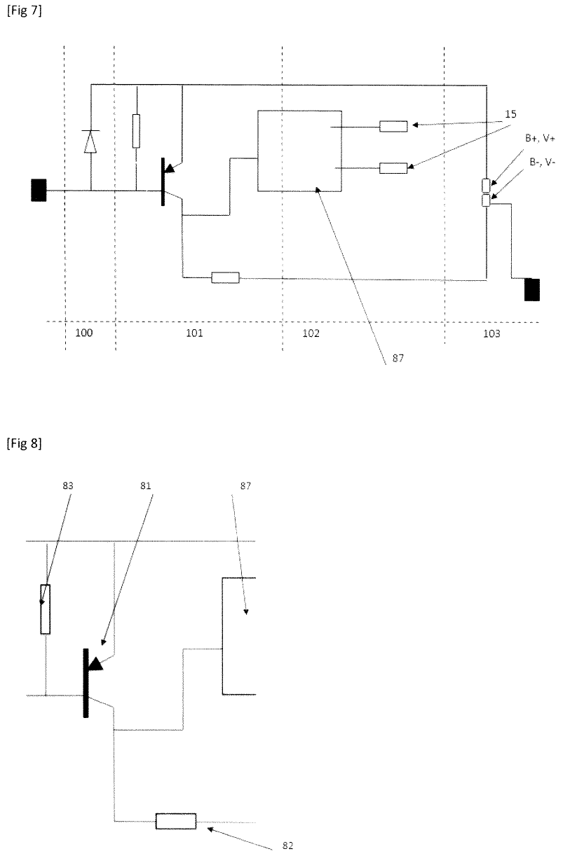

FIG. 7 represents the overall diagram of the intelligent electronic circuit

FIG. 8 represents the diagram which specifies the detection circuit of the intelligent electronic circuit

FIG. 1 shows a view of the two cutlery parts with a cutlery head (1) which includes teeth and a shoulder connecting to the handle and a cutlery handle (2). The two pieces of cutlery joined together reproduce the general shape of a cutlery. They are separated in the immediate vicinity of the shoulder of the utensil head. This solution provides a maximum gripping surface for the handle which will ensure permanent contact with the hand of the cutlery handle (2) when the user holds the cutlery by the handle (2).

FIG. 2 represents a top view of the electronic card (10). The electronic card includes a so-called core circuit (17) with a chip and a dedicated electrical circuit which connects it to the charging and detection elements described later. The core circuit (17) is supplied to the terminals (B+, B−) of a power supply. The card includes 1—a ground contact pad, called the Minus contact pad (12) which is connected directly to the Minus terminal (B−) of circuit 2—a so-called Plus contact pad (11) which is connected to through the core circuit (17) to terminal (B+) 3—warning outputs (15) which are a buzzer (15a) and an LED light (15b) which are connected to the core circuit (17). The function of the warning outputs (17) is to inform the user by means of a vibrating or luminous warning signal whether he is eating too quickly or not.

FIG. 3 represents a side view of the electronic card (10), on which a super capacitor (20) is connected to the power supply terminals (B+, B−); As everyone knows, a super capacitor is a capacitance having the possibility of storing a significant quantity of charges, thus making it possible to release power supply current from the electronic circuit. The novelty being that the super capacities (20) do not still power intelligent cutlery until today for many reasons which are in particular that the charge storages were insufficient for the electricity demand necessary in the design of electronic circuits such as known. Progress being made both in the charging capacity per volume environment and in the design of the electronic circuit itself which, according to our invention, radically reduces consumption, it becomes possible with the combination of these advances to only now design a power supply by super capacity provided that the major trick of recharging the circuit through the two parts of utensil was imagined. In short, the integration of a super capacity within an intelligent utensil is made possible by the combined means of technological progress on the super capacity, technological progress on the electronic circuit and the invention of the integration of a new charging circuit which is compatible with these technological advances themselves. The two contact springs are visible, the Plus terminal contact spring (21) and the Minus terminal contact spring (22) which respectively make contacts by being soldered to the Plus contact pads (11) and the Minus contact pad (12).

FIG. 4 represents a side view of the electronic card inscribed in its protective housing (30). The housing forms a waterproof shell from which only the two contact springs (21, 22) emerge and are flush. A possible solution to create watertightness within the case is to fill the interior of the case with insulating resin.

FIG. 5 represents the assembly of the electronic card in its case with the two utensil parts (1, 2) with the case (30) which attaches to the two parts of the utensil and creates rigidity for maintain the overall shape of a place setting. The specific means of attaching the box for mechanical strength are not described in this patent, but the feasibility is obviously possible by numerous means which are either gluing or mechanical strength by means of hook slides, or mechanical strength using rivets.

FIG. 6 represents the cutlery in its recharging case (50) which includes an external power supply (53) of electrical energy, attached represented by a set of two so-called stick batteries, but which also come from a solar panel. The specificity of the box is that it includes a timing circuit (58) which takes the shape of the utensil and which follows the path of the box contacts (51, 52) which respectively come into contact with one another from the power supply terminals Plus and Minus of the power supply (53) respectively in contact with the utensil head (1) for the housing head contact (51) and with the handle (2) for the housing handle contact (52).

FIG. 7 represents the overall diagram of the intelligent electronic circuit which explains how precisely the definition of the circuit makes the whole as described functional. The card (10) houses from right to left—A power supply circuit (103) with the Plus terminal (B+) at the potential (V+), the terminal (B−) at the potential (V−) in this case at the ground - A warning circuit (102) which includes the warning outputs (15), in this case the buzzer (15a) and the LED light (15b). The warning outputs being driven by an output on the computing chip (87).—A detection circuit (101) described later which detects the mouthing of the utensil head by means in particular of a voltage division circuit on an unregulated power supply, by coupling of pulling resistance, ground resistance and inverted transistor. This circuit makes it possible to detect large resistances and therefore that of the body which connects the fork head to the fork handle when the user brings the fork to the mouth—A charging circuit (100) which cleverly short-circuits the detection circuit (101), in case the charging circuit is powered by an energy source. The short circuit being in this case produced by a diode called a load diode whose threshold value is greater than cleverly calibrated at 0.7 V, that is to say which makes a short circuit on this branch of the circuit if the person is eating and which on the other hand allows the power supply to pass, typically above the voltage of the super capacity plus 0.7 V, and allows the charging of the super capacity, if the cutlery is stored in its case and subjected to a charging voltage.

FIG. 8 represents the diagram which specifies the detection circuit of the intelligent electronic circuit. It must be seen that this circuit does not respond to any other known circuit since its constraint is to detect a very weak voltage variation signal between—a power supply by a super capacitor which is emptying and which therefore offers a voltage which is not stable and which decreases during use—a microcontroller which has its own micro electric properties which can disturb the detection circuit—a charging circuit which must not disturb the measurement. Traditionally, the solution that would be proposed for detecting a short circuit by placing the utensil in the mouth would be to use an operational amp or a comparator. The problem is that these two solutions require a stabilized supply voltage, which is not the case in the case of power supply by super capacity. Clearly, in view of the elements stated above this solution could not be functional because the operational amp would intervene between floating terminals and would therefore not succeed in filtering the events. The circuit must therefore combine the constraints of digital and those of analog by detecting an event between two variable voltages. The proposed solution is to use a so-called pull-up resistor (83) of high value, that is to say in Mega ohm. It is connected between the base of a transistor (81) and the V+ of the super capacitor. The transistor (81) is of the reverse polarity type known as PNP. The pull-up resistor pulls the potential up to avoid false detections. The combination of the pull-up resistor and the human body resistance constitutes a voltage divider connected to the base of the transistor (81). At the transistor output there is a so-called ground resistance (82) of several hundred kilos ohm avoiding disturbing the transistor input stage. The detection input of the calculation chip (87) is connected to the collector of the transistor. The whole thing is functional due to the fine calibration of the resistors surrounding the transistor. These calibrations make it possible to detect the presence of a high value resistance like that of the human body. In a way this part of the circuit works like a very high resistance detector in mega ohm meter. The use of a megohmmeter is impossible since in these devices the supply voltage is stable. And finally, the fact of doing away with a voltage regulator to allow the use of traditional components makes it possible to mix the charging and detection functions. With a voltage regulator it would be impossible to bring the pins together.

The present invention therefore relates to intelligent cutlery to guide the user in their feeding speed produced by assembling a cutlery head (1) of a cutlery handle (2) mechanically assembled with a housing protection (30) of an electronic card (10), the electronic card (10) comprises two power supply terminals (B+, B−) and a Minus contact pad (12) which is connected directly to the terminal supply Minus (B−) of the circuit, a contact pad Plus (11) connected through the core circuit (17) to the terminal (B+), contact springs (21, 22) respectively make contact between the head of utensil (1) and the Plus contact pad (11) and the utensil handle (2) and the contact spring (12) characterized in that the protective box (30) is sealed by a tamper-proof closure, the power supply is housed in the case (20) and is a rechargeable super capacity (20), the cutlery is recharged in a case (50) by positioning, by means of a timing circuit (58) in contact with the cutlery head at the terminal Plus an external power supply (53) and the utensil handle to the Minus terminal of this external power supply (53), the electronic card (10) houses a power supply circuit (103) with the Plus terminal (B+) at potential (V+), terminal (B−) to potential (V−) to ground, and a core circuit (17) which includes a warning circuit (102) which includes the warning outputs (15), with the vibrator (15a) and the LED light (15b), the warning outputs being controlled by an on-chip output of a computing chip (87), a detection circuit (101) which detects mouthing's of the utensil head by means in particular of a voltage division circuit on an unregulated power supply, by coupling of a pull-up resistor (83), a ground resistor (82) and an inverted transistor (81) which is connected to an input of the calculation chip (87), a charging circuit (100) which cleverly short-circuits the detection circuit (101), by means of a diode, in the event that the charging circuit is powered by the source of external energy (53).

1. Intelligent cutlery to guide the user in their feeding speed produced by the assembly of a cutlery head (1) of a cutlery handle (2) mechanically assembled with a protective box (30) of an electronic card (10), the electronic card (10) comprises two power supply terminals (B+, B−) and a Minus contact pad (12) which is connected directly to the Minus power terminal (B−) of the circuit, a Plus contact pad (11) connected through the core circuit (17) to the terminal (B+), contact springs (21, 22) respectively make contact between the utensil head (1) and the Plus contact pad (11) and between the utensil handle (2) and the Minus contact pad (12) characterized in that the protective housing (30) is sealed by a tamper-proof closure, the power supply is housed in the protective box (30) and is a rechargeable super capacity (20), the utensil is recharged in a case (50) by positioning by means of a timing circuit (58) in contact the utensil head with the Plus terminal of an external power supply (53) and the utensil handle to the Minus terminal of this external power supply (53).

2. Intelligent utensil for guiding the user in his feeding speed according to claim 1 characterized in that the core circuit (17) of the electronic card (10) comprises a warning circuit (102) with warning outputs (15), buzzer (15a), LED light (15b), the warning outputs being driven by an on-chip output of a computing chip (87), a detection circuit (101) which detects the mouthing of the cutlery head by means of a voltage division circuit on an unregulated power supply, by coupling of a pulling resistance (83), a ground resistance (82) and an inverted transistor (81) which is connected to an input of the calculation chip (87), a charging circuit (100) which, by means of a diode at a threshold lower than the charging voltage, cleverly short-circuits the detection circuit (101), in case where the charging circuit is powered by the external energy source (53).)
